(19) United States
(12) Patent Application Publication
(10) Pub. No.: US 2023/0081064 A1
(43) Pub. Date: Mar. 16, 2023

(54) FORMULATIONS FOR DELIVERY TO THE EAR

(71) Applicant: Spiral Therapeutics Inc., South San Francisco, CA (US)

(72) Inventors: Kathleen Farinas, South San Francisco, CA (US); Andrew Ayoob, San Francisco, CA (US)

(21) Appl. No.: 17/897,944

(22) Filed: Aug. 29, 2022

Related U.S. Application Data
(60) Provisional application No. 63/239,588, filed on Sep. 1, 2021.

(51) Int. Cl. A61K 47/10; A61K 47/32; A61K 45/06; A61K 9/06; A61K 47/02

(52) U.S. Cl. CPC A61K 47/10; A61K 47/32; A61K 45/06; A61K 9/06; A61K 47/02

(57) ABSTRACT

Disclosed herein are polymer compositions comprising, in some cases, about 15% to about 22% by weight of poloxamer 407, about 1% to about 12% by weight of poloxamer 188, and about 3% to about 15% by weight of PEG 300, wherein the total amount of poloxamer 188 and PEG300 in the composition is about 12% to about 25% by weight. In some cases, polymer compositions disclosed herein can be used for delivery of an active agent to the ear of a subject.

### CROSS-REFERENCE TO RELATED APPLICATIONS

This application claims benefit of priority to U.S. Provisional Application No. 63/239,588, filed on Sep. 1, 2021. The contents of which are hereby incorporated by reference in their entirety.

### TECHNICAL FIELD

The present disclosure relates to polymer compositions for delivery, for example, of one or more active agents, to the ear. Typically, the polymer compositions disclosed herein form a gel at or around body temperature.

### BACKGROUND

Otic diseases and disorders can be challenging to treat, as it may be beneficial, or even essential, to deliver active agents to the middle and/or inner ear. Non-limiting examples of such disorders or conditions include Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, and similar ear disorders and conditions. Formulating active agents (e.g., drugs) for delivery to the middle and/or inner ear can be challenging, due to the properties of the active agent(s), the location of delivery, or both. In some cases, a sustained release system can be useful, as delivery of the active agent(s), such as through intratympanic injection, can be intimidating or uncomfortable for a subject and cannot be self-administered.

Ménière's Disease is a chronic, incurable inner ear disorder with recurrent debilitating symptoms that affect hearing and balance. It is named for French physician Prosper Ménière who, in 1861, first identified and described the symptoms of this medical condition. Researchers are unsure of what causes the buildup of fluid in the inner ear that results in MD. Some believe it is related to vascular insufficiencies, others say it might be due to autoimmune conditions, viral infections, allergic reactions or that the disease may initiate from a trauma. In some cases, MD appears to have a hereditary component, so a gene mutation may be connected to the regulation of inner ear fluid.

Autoimmune Inner Ear Disease is a rare disorder, appearing in both adults and children, caused by an immune system response. The inner ear can be the direct target of the immune response, but it can be additionally damaged by a deposition of circulating immune complexes or by systemic immune-mediated diseases. The clinical expression of immune-mediated inner ear disease can show a progressive bilateral and asymmetric sensorineural hearing loss (SNHL) profile. Cochlear symptoms are often associated with vestibular disorders. In about 50% of AIED patients, hearing loss is also associated with vestibular symptoms, such as imbalance and motion intolerance, ataxia and positional or episodic vertigo.

Sensorineural Hearing Loss is due to impaired ability of the cochlea to effectively transduce pressure waves into neural signaling. SNHL is typically associated with exposure to loud noise, aging, head trauma, exposure to ototoxic drugs, infection, autoimmune disease, Ménière's disease, genetic mutations, and tumors of the auditory nerve.

Noise-induced hearing loss is caused by exposure to loud and/or long-lasting sounds. Hearing loss-may occur from prolonged exposure to loud noises, such as heavy machinery, loud music, airplanes or gunfire. Long, repeated or impulse exposure to sounds at or above 85 decibels can cause hearing loss. NIHL causes damage to the hair cells and/or the auditory nerve.

Symptoms of MD, AIED, SNHL, NIHL, and other ear disorders can include vertigo, hearing loss, ear ringing (tinnitus), and/or ear pressure. In some cases, the vertigo may cause severe nausea and imbalance. Hearing loss may become permanent. There is no treatment, other than for some of the symptoms, nor a cure. Drugs for motion sickness or nausea may help manage the symptoms.

Accordingly, provided herein are polymer compositions suitable for delivery (e.g., of an active agent) to the middle and/or inner ear.

### SUMMARY

In general, provided herein are polymer compositions useful for administration (e.g., of an active agent) to the middle and/or inner ear. In some cases, a polymer composition provided herein can be stable (e.g., displaying no precipitation of an active agent) for days, weeks, months, or years, at cool and/or ambient conditions. The polymer compositions provided herein typically form a gel (e.g., a hydrogel) at or around body temperature.

Provided herein is a polymer composition comprising about 15% to about 22% by weight of poloxamer 407, about 1% to about 12% by weight of poloxamer 188, and about 3% to about 15% by weight of PEG 300, wherein the total amount of poloxamer 188 and PEG300 in the composition is about 12% to about 25% by weight.

Also provided herein is a polymer composition comprising about 15% to about 22% by weight of poloxamer 407, about 1% to about 12% by weight of poloxamer 188, and about 3% to about 15% by weight of PEG 300, provided that (a) if the amount of poloxomer 188 is less than 3% by weight, the amount of PEG 300 is about 13% to about 15% by weight, (b) if the amount of poloxamer 188 is about 3% to about 5% by weight and the amount of poloxamer 407 is less than about 17% by weight, the amount of PEG 300 is about 13% to about 15% by weight, (c) if the amount of poloxamer 188 is about 3% to about 5% by weight and the amount of poloxamer 407 is at least about 17% by weight, the amount of PEG 300 is about 9% to about 15% by weight, and (d) if the amount of poloxamer 188 is about 10% to about 12% by weight, the amount of PEG 300 is about 3% to about 10% by weight.

In some embodiments, the total amount of poloxamer 188 and PEG300 in the composition is about 12% to about 25% by weight. In some embodiments, the total amount of poloxamer 188 and PEG300 in the composition is about 12% to about 22% by weight. In some embodiments, the total amount of poloxamer 188 and PEG300 in the composition is about 19% to about 20% by weight. In some embodiments, the amount of poloxamer 407 is about 16% to about 19% by weight. In some embodiments, the amount of poloxamer 407 is about 16% to about 17% by weight. In some embodiments, the amount of poloxamer 407 is about 17% to about 18% by weight. In some embodiments, wherein the amount of poloxamer 407 is about 18% to about 19% by weight. In some embodiments, the amount of poloxamer 188 is about 2.5% to about 12% by weight.

In some embodiments, the polymer composition of any one the compositions disclosed herein the amount of poloxamer 188 is about 1% to about 3% by weight. In some embodiments, the amount of PEG 300 is about 13% to about 15% by weight. In some embodiments, the amount of poloxamer 188 is about 3% to about 5% by weight. In some embodiments, the amount of poloxamer 407 is less than about 17% by weight and the amount of PEG 300 is about 13% to about 15% by weight. In some embodiments, the amount of poloxamer 407 is at least about 17% by weight and the amount of PEG 300 is about 9% to about 15% by weight. In some embodiments, the amount of poloxamer 407 is at least about 17% by weight and the amount of PEG 300 is about 9% to about 13% by weight. In some embodiments, the amount of poloxamer 188 is greater than about 5% and less than about 10% by weight. In some embodiments, the amount of PEG 300 is about 3% to about 7% by weight. In some embodiments, the amount of PEG 300 is about 7% to about 11% by weight. In some embodiments, the amount of PEG 300 is about 9% to about 13% by weight. In some embodiments, the amount of poloxamer 188 is about 10% to about 12% by weight. In some embodiments, the amount of PEG 300 is about 3% to about 10% by weight. In some embodiments, the amount of PEG 300 is about 3% to about 7% by weight. In some embodiments, the amount of PEG 300 is about 7% to about 10% by weight.

In some embodiments, any of the polymer compositions described herein comprises about 0.01% to about 20% by weight of an active agent. In some embodiments, the polymer compositions described herein comprise about 0.01% to about 1.0% by weight of an active agent.

Also provided herein is a polymer composition comprising: about 17% to about 20% by weight of poloxamer 407, about 9% to about 11% by weight of poloxamer 188, about 8% to about 10% by weight of PEG 300, and about 0.01% to about 1.0% by weight of an active agent. Also provided herein is a polymer composition comprising about 18.5% by weight of poloxamer 407, about 10.0% by weight of poloxamer 188, about 9.0% by weight of PEG 300, and about 0.01% to about 1.0% by weight of an active agent.

In some embodiments, the active agent has a solubility in water of less than about 100 μM. In some embodiments, the active agent has a solubility in water of less than about 50 μM. In some embodiments, the active agent has a solubility in water of less than about 25 μM. In some embodiments, the active agent has a solubility in water of less than about 10 μM. In some embodiments, the active agent has a solubility in water of less than about 5 μM. In some embodiments, the active agent has a solubility in water of less than about 2.5 μM. In some embodiments, the active agent has a melting temperature of at least 125° C. In some embodiments, the active agent has a melting temperature of at least 150° C. In some embodiments, the active agent has a melting temperature of at least 175° C. In some embodiments, the active agent has a melting temperature of at least 200° C.

In some embodiments, any one of the polymer compositions described herein comprise about 0.03% to about 0.07% by weight of the active agent. In some embodiments, any one of the polymer compositions described herein comprise about 0.05% by weight of the active agent. In some embodiments, any one of the polymer compositions described herein comprise about 0.5% to about 3% of polyvinylpyrrolidone. In some embodiments, any one of the polymer compositions described herein comprise water. In some embodiments, any one of the polymer compositions described herein comprise about 0.5 mM to about 10 mM of a buffer. In some embodiments, any one of the polymer compositions described herein comprise about 1 mM to about 3 mM of a buffer.

In some embodiments, the buffer is selected from the group consisting of a phosphate buffer, a tris(hydroxymethyl)aminomethane buffer, a citrate buffer, an acetate buffer, a 4-(2-hydroxyethyl)-1-piperazineethanesulfonic acid buffer, or a combination thereof. In some embodiments, the buffer is a phosphate buffer. In some embodiments, any one of the polymer compositions described herein comprise about 1 mM to about 15 mM of a salt. In some embodiments, any one of the polymer compositions described herein comprise about 5 mM to about 10 mM of a salt. In some embodiments, the salt is selected from the group consisting of sodium chloride, potassium chloride, calcium chloride, magnesium chloride, sodium thiosulfate, sodium bisulfate, sodium bicarbonate, ammonium sulfate, and combinations thereof. In some embodiments, the salt is sodium chloride.

In some embodiments, the active agent is physically stable in the composition for at least 14 days at 4° C. In some embodiments, the active agent is physically stable in the composition for at least 56 days at 4° C. In some embodiments, the active agent is physically stable in the composition for at least 4 months at 4° C. In some embodiments, the active agent is physically stable in the composition for at least 6 months at 4° C. In some embodiments, the active agent is physically stable in the composition for at least 1 year at 4° C. In some embodiments, the active agent is physically stable in the composition for at least 2 years at 4° C.

In some embodiments, the polymer composition has a degree of supersaturation of the active agent of about 1.0 to about 2.5 after 1 day at 15° C. In some embodiments, the polymer composition has a degree of supersaturation of the active agent of about 1.0 to about 2.0 after 1 day at 15° C. In some embodiments, the polymer composition has a degree of supersaturation of the active agent of about 1.0 to about 1.5 after 1 day at 15° C. In some embodiments, the polymer composition has a degree of supersaturation of the active agent of about 1.0 to about 2.5 after 1 day at 4° C. In some embodiments, the polymer composition has a degree of supersaturation of the active agent of about 1.0 to about 2.0 after 1 day at 4° C.

In some embodiments, the polymer composition has a degree of supersaturation of the active agent of about 1.0 to about 1.5 after 1 day at 4° C. In some embodiments, the polymer composition forms a gel at a temperature of about 24° C. to about 32° C. In some embodiments, the polymer composition forms a gel at a temperature of about 26° C. to about 30° C.

Also provided herein is a gel formed by any of the polymer compositions described herein.

Also provided herein is a manufacture of a medicament for the treatment of an otic disease or disorder comprising any one of the polymer compositions described herein.

Also provided herein is a method of preparing any of the polymer compositions described herein the method comprising dissolving the active agent in the PEG 300 to form an active agent solution; and adding to the active agent solution to an aqueous solution comprising the poloxamer 407, and the poloxamer 188.

Also provided herein is a method of treating an otic disease or disorder in a subject in need thereof, the method comprising administering to an affected ear of the subject a therapeutically effective amount of any of the polymer compositions described herein.

Also provided herein is a method of treating an otic disease or disorder in a subject, the method comprising (a) identifying the subject as having the otic disease or disorder; and (b) administering to an affected ear of the subject a therapeutically effective amount of any one of the polymer compositions described herein.

In some embodiments, the method further comprises, before the administering, preparing the polymer composition. In some embodiments, preparing the polymer composition comprises: dissolving the active agent in the PEG 300 to form an active agent solution; and adding to the active agent solution an aqueous solution comprising the poloxamer 407, and the poloxamer 188.

In some embodiments, the otic disease or disorder is selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof.

The details of one or more embodiments of the disclosure are set forth in the accompanying drawings and the description below. Other features, objects, and advantages of the disclosure will be apparent from the description and drawings, and from the claims. The word “comprising” in the claims may be replaced by “consisting essentially of” or with “consisting of,” according to standard practice in patent law.

### BRIEF DESCRIPTION OF THE DRAWINGS

FIG. 1 is a schematic of polymer components of a polymer composition, as well as exemplary challenges at various combined concentrations.

FIG. 2A is a plot of the percent cumulative release of an active agent from an exemplary gelled polymer composition disclosed herein, plotted against time in hours.

FIG. 2B is a plot of the percent cumulative release of an active agent from an exemplary gelled polymer composition disclosed herein, plotted against the square root of time in hours.

FIG. 2C is a plot of the comparative percent cumulative release of an active agent from an exemplary gelled polymer composition disclosed herein and a control polymer composition, plotted against time in hours.

FIG. 3 is a plot of the viscosity versus temperature for exemplary polymer compositions disclosed herein.

### DETAILED DESCRIPTION

### I. Definitions

“Active agent” and “active pharmaceutical ingredient” are used interchangeably and refer to a physiologically or pharmacologically active substance that acts locally and/or systemically in the body. An active agent is a substance that is administered to a patient for the treatment, prevention, or diagnosis of a disease or disorder.

The term “blood labyrinth barrier” or “BLB” refers to the barrier between the vasculature and the inner ear fluids, either endolymph or perilymph. The BLB is involved in the maintenance of the inner ear fluid ionic homeostasis.

The phrase “effective amount” or “effective concentration” means an amount of active agent that, when at a site of action, is sufficient to (i) treat a disease, condition, or disorder, (ii) attenuate, ameliorate, or eliminate one or more symptoms of the particular disease, condition, or disorder, or (iii) delay the onset of one or more symptoms of the particular disease, condition, or disorder described herein. The amount of an active agent that will correspond to such an amount will vary depending upon factors such as the particular active agent, the particular disease and its severity, and the identity (e.g., age and/or weight) of the patient in need of treatment, but can nevertheless be routinely determined by one skilled in the art. The terms “effective amount” or “therapeutically effective amount,” as used herein, can refer to a sufficient amount of an active agent at a site of action that would be expected to relieve to some extent one or more of the symptoms of the disease or condition being treated. In some embodiments, an effective amount of an active agent is a quantity necessary to render a desired anti-inflammatory result at a site of action. The term “therapeutically effective amount” includes, for example, an “effective amount” of an active agent to achieve a desired pharmacologic effect without undue adverse side effects.

The phrase “effective dose” means an amount of active agent that, when administered to a patient in need of such treatment, is sufficient to (i) treat a disease, condition, or disorder, (ii) attenuate, ameliorate, or eliminate one or more symptoms of the particular disease, condition, or disorder, or (iii) delay the onset of one or more symptoms of the particular disease, condition, or disorder described herein. In some embodiments, an “effective dose” is an amount of active agent, when administered to a patient in need of such treatment, that achieves a sufficient concentration at a site of action to (i) treat a disease, condition, or disorder, (ii) attenuate, ameliorate, or eliminate one or more symptoms of the particular disease, condition, or disorder, or (iii) delay the onset of one or more symptoms of the particular disease, condition, or disorder described herein for a period of time. The dose of an active agent that will correspond to such an amount will vary depending upon factors such as the particular active agent, the particular disease and its severity, and the identity (e.g., age and/or weight) of the patient in need of treatment, but can nevertheless be routinely determined by one skilled in the art. The terms “effective dose” or “therapeutically effective dose,” as used herein, can refer to a sufficient amount of an active agent being administered that would be expected to relieve to some extent one or more of the symptoms of the disease, condition, or disorder being treated. In some embodiments, an effective dose of an active agent is a quantity necessary to render a desired anti-inflammatory result. The term “therapeutically effective dose” includes, for example, an “effective dose” of an active agent to achieve a desired pharmacologic effect without undue adverse side effects. It will also be understood that “an effective dose” in an extended-release dosing format may differ from “an effective dose” in an immediate-release dosing format based upon pharmacokinetic and/or pharmacodynamic considerations.

The term “MRSD” or “maximum recommended starting dose” refers to the highest amount of an agent that can be given safely and without complication while maintaining its efficacy.

The term “MTD” or “maximum tolerated dose” refers to the highest dose of a drug or prevention that does not cause unacceptable side effects.

The term “NOAEL” refers to “no observed adverse effect level” and is an important part of the non-clinical risk assessment.

The terms “otic” and “auris” refer to relating to the ear. For example, an otic composition can be a composition intended for administration to the ear.

The term “pharmaceutically acceptable” indicates that the compound, or salt or composition thereof is compatible chemically and/or toxicologically with the other ingredients comprising a formulation and/or the patient being treated therewith. In some embodiments, a pharmaceutically acceptable salt can be a salt that conserves the efficiency and/or the biological properties of the free bases or free acids. In some embodiments, a pharmaceutically acceptable salt can be a salt that change the efficiency and/or the biological properties of the free bases or free acids; for example, a pharmaceutically acceptable salt can improve the bioavailability of a free base or free acid.

The term “prophylactically effective amount” or “prophylactically effective dose” means an amount of active agent that, when administered to a patient in need of such treatment, is sufficient to (i) prevent a disease, condition, or disorder, (ii) attenuate, ameliorate, or eliminate one or more symptoms of the particular disease, condition, or disorder, before it occurs. In some cases, a “prophylactically effective amount” refers to an amount of a composition administered to a subject susceptible to or otherwise at risk of a particular disease, disorder, or condition, for example, a prophylactically effective amount of an active agent can be an amount effective to prevent or to attenuate ototoxicity. For example, an apoptotic inhibitory formulation may be administered to an individual prior to chemotherapy to prevent hearing loss by a subsequently administered chemotherapeutic agent.

The term “residence time” as used herein can refer to the amount of time that a formulation remains in the location of administration. In some embodiments, residence time can be the time when there is no gel visualized on the round window membrane area, e.g., after collecting the gel at a time after injection.

The term “room temperature” refers to a temperature between about 15° C. and less than about 27° C., for example about 25° C. or about 20° C.

The term “body temperature” refers to a temperature between about 36.5° C. and about 37.5° ° C., preferably about 37° C.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease, condition, or disorder to be treated and/or prevented.

“Stable” as used herein can refer to chemical and/or physical stability over a time period under defined conditions. In some embodiments, a physically stable solution can retain a high percentage or all of what was originally dissolved remaining in solution. In some embodiments, a physically stable solution can retain more than 60, 70, 80, 90, 95, 98, 99, or 100% of the originally dissolved solute(s) at room temperature (approximately 15-25° C., most preferably 25° C.). Physical stability of a polymer composition (e.g., including an active agent) can be determined using any appropriate method. One method is to monitor the appearance of a polymer composition including an active agent for turbidity, precipitation, or both, for example, using visual inspection (e.g., under 5× or higher magnification (e.g., with microscopy)). As another example, physical stability of a solution can be determined by filtering the solution (e.g., removing precipitates) and measuring the concentration of one or more dissolved solutes. Another method of measuring physical stability of a solution is to use light scattering, which can detect the presence of particulate matter in a solution.

“Sustained release” as used herein refers to release of a substance over an extended period of time. In some embodiments, this can be contrasted with a bolus type administration (e.g., immediate release) in which the entire amount of the substance is made biologically available at one time.

The term “transtympanic administration” refers to the administration of an active agent (e.g., a therapeutic agent) via the tympanic cavity, preferably via a needle that accesses the tympanic cavity (middle ear) by penetrating the tympanic membrane (eardrum).

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (e.g., not worsening) state of disease, delay or slowing of disease progression, amelioration or palliation of the disease state (e.g., one or more symptoms of the disease), and remission (whether partial or total), whether detectable or undetectable.

The term “unit dosage form” refers to physically discrete units suitable as unitary dosages for human subjects and other patients, each unit containing a predetermined quantity of active material (e.g., an active agent as provided herein) calculated to produce the desired therapeutic effect, in association with a suitable pharmaceutical excipient.

The terms “prevent,” “preventing” or “prevention,” as used herein means the prevention of the onset, recurrence or spread, in whole or in part, of a disease, disorder, or condition as described herein, or a symptom thereof.

### II. Polymer Compositions

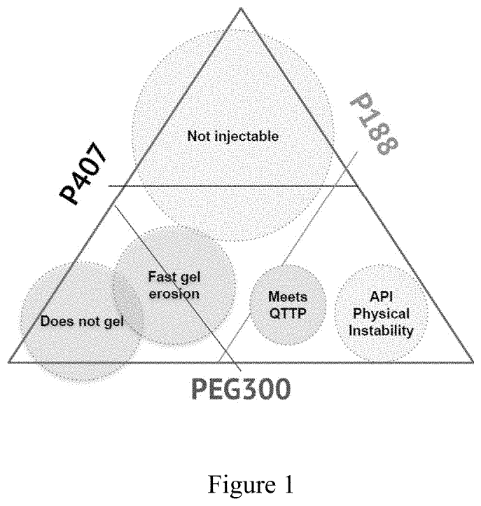

Polymer compositions as provided herein are typically suitable for formulating an active agent (e.g., a poorly soluble active agent) for administration to the middle and/or inner ear. Polymer molecules within a polymer composition can form micelles which can increase the overall solubilization and promote physical stability. In some incidences, polymer compositions including one or more poloxamers and polyethylene glycol can be an injectable liquid at a first temperature and gel at a second temperature. For example, it has been surprisingly found that particular ranges of poloxamer 407, poloxamer 188, and polyethylene glycol 300, when combined, yield a polymer composition that is injectable, forms a gel (e.g., a gel with desirable erosion properties), and in which an active agent is physically stable in solution. FIG. 1 shows a schematic of these three components in polymer compositions.

Accordingly, described herein are polymer compositions including one or more poloxamers and polyethylene glycol. In some examples the polyethylene glycol is a low molecular weight polyethylene glycol. Examples of low molecular weight polyethylene glycol include polyethylene glycol 200 (PEG200), polyethylene glycol 300 (PEG300), polyethylene glycol 400 (PEG400), polyethylene glycol 500 (PEG500), polyethylene glycol 600 (PEG600), polyethylene glycol 700 (PEG700), and polyethylene glycol 800 (PEG800).

The temperature at which a polymer composition transitions to a gel is called a critical solution temperature. In some embodiments described herein, are polymer compositions including poloxamer 407 (also called P407), poloxamer 188 (also called P188), and polyethylene glycol 300 (also called PEG300). In some embodiments, polymer compositions described herein can include additional components, for example, a crystallization inhibitor, a buffer, a salt, or a combination thereof.

In general, poloxamers are non-ionic surfactants that are synthetic tri-block copolymers composed of a central hydrophobic chain of polyoxypropylene flanked by two hydrophilic chains of polyoxyethylene with an approximate weight ratio of 4:2:4. Poloxamers are commonly abbreviated with a letter “P” (for poloxamer) followed by three digits. Typically, the first two digits multiplied by 100 give the approximate molecular mass of the polyoxypropylene core and the last digit multiplied by 10 gives the approximate percentage of polyoxyethylene content.

Poloxamer 407 displays reversible thermogelling properties and can be considered to be a primary thermogelling agent of the polymer compositions disclosed herein. Above the critical solution temperature for a given P407 concentration, micelles pack together, creating a gel at a higher temperature (the “gelation temperature”). Poloxamer 407 may also increase active agent solubility through association of drug molecules with the hydrophobic blocks in the P407 polymer below the critical solution temperature, increase incorporation of active agent into the hydrophobic micelle cores above the critical solution temperature, or both. In the polymer compositions herein, P188 helps to lower the critical solution temperature and helps to raise the gelation temperature of the polymer compositions. One benefit of including PEG300 in the polymer compositions is that some active agents (e.g., poorly soluble active agents (e.g., LPT99)) can be dissolved in PEG300, whereas they may not be able to be solubilized in the other components without additional steps, such as using ultrasound.

Surprisingly, it has been found that these three components, at certain ratios, work together to produce polymer compositions that are suited to delivery (e.g., of an active agent) to the ear. In some cases, they are particularly well suited to poorly-soluble active agents (e.g., LPT99). In Example 2, it is shown that PEG300 alone with P407 was able to dissolve the active agent and remain physically stable in solution for about 6 days, but together with P188, the solution was clear after 14 days at 2-8° C.

Accordingly, provided are polymer compositions including P407, P188, and PEG300. In some embodiments, the polymer compositions can include about 15% to about 22% by weight of poloxamer 407; about 1% to about 12% by weight of poloxamer 188; about 3% to about 15% by weight of PEG 300; wherein the total amount of poloxamer 188 and PEG300 in the composition is about 12% to about 25% by weight.

In some embodiments, the polymer compositions can include about 15% to about 22% by weight of poloxamer 407; about 1% to about 12% by weight of poloxamer 188; and about 3% to about 15% by weight of PEG 300; provided that: (a) if the amount of poloxomer 188 is less than 3% by weight, the amount of PEG 300 is about 13% to about 15% by weight, (b) if the amount of poloxamer 188 is about 3% to about 5% by weight and the amount of poloxamer 407 is less than about 17% by weight, the amount of PEG 300 is about 13% to about 15% by weight, (c) if the amount of poloxamer 188 is about 3% to about 5% by weight and the amount of poloxamer 407 is at least about 17% by weight, the amount of PEG 300 is about 9% to about 15% by weight, and (d) if the amount of poloxamer 188 is about 10% to about 12% by weight, the amount of PEG 300 is about 3% to about 10% by weight. In some such embodiments, the total amount of poloxamer 188 and PEG300 in the composition is about 12% to about 25% by weight.

In some embodiments of any of the polymer compositions, the total amount of poloxamer 188 and PEG300 in the composition can be about 12% to about 22% by weight or about 19% to about 20% by weight.

In some embodiments of any of the polymer compositions, the poloxamer 407 can be present in an amount of about 15% to about 22% by weight of the polymer composition, such as about 16% to about 19%, about 16% to about 17%, about 17% to about 18%, or about 18% to about 19% by weight.

In some embodiments of any of the polymer compositions, the poloxamer 188 can be present in an amount of about 1% to about 12% by weight of the polymer composition, such as about 2.5% to about 12%, about 1% to about 3%, about 3% to about 5%, greater than about 5% and less than about 10%, or about 10% to about 12% by weight.

In some embodiments of any of the polymer compositions, the PEG300 can be present in an amount of about 3% to about 15% by weight of the polymer composition, such as about 3% to about 7%, about 3% to about 10%, about 7% to about 10%, about 7% to about 11%, about 9% to about 13%, about 9% to about 15%, or about 13% to about 15% by weight.

Without being bound by any particular theory, it is believed that the amounts of the poloxamer 407, poloxamer 188, and PEG300 components can be dependent on one another (e.g., to make a polymer composition suitable for delivery (e.g., of an active agent) to the middle and/or inner ear. For example, if the amount of poloxamer 188 is about 1% to about 3% (e.g., about 2.5% to about 3%) by weight of the polymer composition, then about 13% to about 15% by weight of PEG300 can be chosen to complement that amount of poloxamer 188. As another example, if the amount of poloxamer 188 is about 3% to about 5% of the polymer composition, and if the amount of poloxamer 407 is less than about 17% weight, then the amount of PEG300 can be about 13% to about 15% by weight. As yet another example, if the amount of poloxamer 188 is about 3% to about 5% of the polymer composition, and if the amount of poloxamer 407 is at least about 17% weight, then the amount of PEG300 can be about 9% to about 15% by weight (e.g., about 9% to about 13% by weight). In some cases, if the amount of poloxamer 188 is greater than about 5% and less than about 10% by weight, then the amount of PEG300 can be about 3% to about 7% by weight, about 7% to about 11% by weight, or about 9% to about 13% by weight. In some embodiments, if the amount of poloxamer 188 is about 10% to about 12% by weight, then the amount of PEG300 can be about 3% to about 10% by weight (e.g., about 3% to about 7% by weight, or about 7% to about 10% by weight).

In some embodiments, provided herein is a polymer composition comprising about 17% to about 20% by weight of poloxamer 407, about 9% to about 11% by weight of poloxamer 188, and about 8% to about 10% by weight of PEG 300. In some embodiments, provided herein is a polymer composition comprising about 18.5% by weight of poloxamer 407, about 10.0% by weight of poloxamer 188, and about 9.0% by weight of PEG 300.

Any of the polymer compositions provided herein can further include an active agent. In some embodiments, an active agent can be present in a polymer composition from about 0.01% to about 20% (e.g., about 0.01% to about 1%, about 0.01% to about 5%, about 0.01% to about 10%, about 0.01% to about 15%, about 1% to about 20%, about 5% to about 20%, about 10% to about 20%, or about 15% to about 20%) by weight of the polymer composition. In some embodiments, an active agent can be present in a polymer composition from about 0.001% to about 5% (e.g., about 0.001% to about 0.005%, about 0.001% to about 0.01%, about 0.001% to about 0.05%, about 0.001% to about 0.1%, about 0.001% to about 0.5%, about 0.001% to about 1%, about 0.001% to about 2%, about 0.001% to about 3%, about 0.001% to about 4%, about 0.005% to about 5%, about 0.01% to about 5%, about 0.05% to about 5%, about 0.1% to about 5%, about 0.5% to about 5%, about 1% to about 5%, about 2% to about 5%, about 3% to about 5%, about 4% to about 5%, about 0.01% to about 1.0%, or about 0.03% to about 0.07%) by weight of the polymer composition. In some embodiments, an active agent can be present in the polymer composition in an amount of about 0.03% to about 0.07%, or about 0.05% by weight.

In some embodiments, provided herein is a polymer composition comprising about 17% to about 20% by weight of poloxamer 407, about 9% to about 11% by weight of poloxamer 188, about 8% to about 10% by weight of PEG 300, and about 0.015 to about 1.0% by eight of an active agent. In some embodiments, provided herein is a polymer composition comprising about 18.5% by weight of poloxamer 407, about 10.0% by weight of poloxamer 188, about 9.0% by weight of PEG 300, and about 0.01% to about 1.0% by weight of an active agent.

An active agent can be any appropriate active agent. The polymer compositions provided herein may be particularly advantageous for active agents that are poorly soluble in water. For example, in some embodiments, the active agent can have a solubility in water of less than about 100 μM (e.g., less than 50 μM, 25 μM, 10 μM, 5 μM, or 2.5 μM). Without being bound by any particular theory, it is believed that when in a solid (e.g., crystalline) form, solubility of an active agent is correlated melting point or melting temperature; e.g., a more stable crystal has lower solubility. Melting temperature can be determined by any appropriate method, for example, use of a differential scanning calorimeter can be used to measure the temperature of the endothermic peak associated with melting the active agent; or melting point can be measured in a melting point apparatus where the active agent is heated and the sample is observed to determine the temperature where the phase change from solid to liquid occurs. In some embodiments, an active agent disclosed herein has a melting temperature greater than about 125° C. (e.g., greater than about 150° ° C., 175° C., or 200° C.). As another example, the active agent can be LPT99 (2-(4-(2,4-Dichlorophenethyl)-3,6-dioxo-1-(2-(thiophen-2-yl)ethyl)piperazin-2-yl)-N-(2-(5-methoxy-1H-indol-3-yl)ethyl)acetamide), which has a water solubility of about 2.4 μM. Other non-limiting examples of active agents include neuroprotective agents, steroids, diuretics, vasodilators, antiinfectives or antihistamines.

In some embodiments, a polymer composition can include a crystallization inhibitor. A non-limiting example of a crystallization inhibitor is polyvinylpyrrolidone (PVP). In some embodiments, a polymer composition can include about 0.1% to about 15% (e.g., about 0.1% to about 0.5%, about 0.1% to about 1%, about 0.1% to about 2%, about 0.1% to about 3%, about 0.1% to about 5%, about 0.1% to about 10%, about 0.5% to about 15%, about 1% to about 15%, about 2% to about 15%, about 3% to about 15%, about 5% to about 15%, about 10% to about 15%, or about 0.5% to about 3%) by weight of a crystallization inhibitor (e.g., PVP). In some embodiments, a polymer composition can include about 0.1% to about 5% (e.g., about 0.5% to about 3%) by weight of a crystallization inhibitor (e.g., PVP). In some embodiments, a polymer composition can include no crystallization inhibitor.

In some embodiments, a polymer composition can include a buffer. A buffer can be any auris-acceptable buffer. Non-limiting examples of auris-acceptable buffers include alkali or alkaline earth metal carbonates, phosphates, bicarbonates, citrates, borates, acetates, succinates and the like, such as sodium phosphate, citrate, borate, acetate, bicarbonate, carbonate and tromethamine (tris(hydroxymethyl)aminomethane, also called TRIS). In some embodiments, the buffer can be selected from the group consisting of a phosphate buffer, a tris(hydroxymethyl)aminomethane buffer, a citrate buffer, an acetate buffer, a 4-(2-hydroxyethyl)-1-piperazineethanesulfonic acid (HEPES) buffer, or a combination thereof. In some embodiments, a buffer can be a phosphate buffer. A buffer can be present in a polymer composition at any appropriate concentration, for example, about 0.1 mM to about 10 mM. In some embodiments, a buffer can be present in a polymer composition at a concentration of about 0.1 mM to about 10 mM (e.g., about 0.1 mM to about 0.5 mM, about 0.1 mM to about 1 mM, about 0.1 mM to about 2 mM, about 0.1 mM to about 3 mM, about 0.1 mM to about 5 mM, about 0.5 mM to about 10 mM, about 1 mM to about 10 mM, about 2 mM to about 10 mM, about 3 mM to about 10 mM, about 5 mM to about 10 mM, about 0.5 mM to about 5 mM, or about 1 mM to about 3 mM). In some embodiments, a buffer can be present in a polymer composition in a concentration of about 0.5 mM to about 5 mM (e.g., about 1 mM to about 3 mM (e.g., about 1.7 mM)). In some embodiments, a polymer composition, or a gel formed by a polymer composition, can have a pH of about 3 to about 10 (e.g., about 3 to about 5, about 3 to about 7, about 3 to about 9, about 5 to about 10, about 7 to about 10, about 9 to about 10, about 5 to about 8, or about 6 to about 8).

In some embodiments, a polymer composition can include a salt. A salt can be any auris-acceptable salt. Non-limiting examples of auris-acceptable salts include those having sodium, potassium or ammonium cations and chloride, citrate, ascorbate, borate, phosphate, bicarbonate, sulfate, thiosulfate or bisulfate anions; suitable salts include sodium chloride, potassium chloride, sodium thiosulfate, sodium bisulfite and ammonium sulfate. In some embodiments, a salt can be selected from group consisting of sodium chloride, potassium chloride, calcium chloride, magnesium chloride, sodium thiosulfate, sodium bisulfite, sodium bicarbonate, ammonium sulfate, and combinations thereof. In some embodiments, the salt is sodium chloride. A salt can be present in a polymer composition at any appropriate concentration, for example, about 0.5 mM to about 25 mM. In some embodiments, a salt can be present in a polymer composition at a concentration of about 0.5 mM to about 25 mM (e.g., about 0.5 mM to about 1 mM, about 0.5 mM to about 2 mM, about 0.5 mM to about 3 mM, about 0.5 mM to about 5 mM, about 0.5 mM to about 10 mM, about 0.5 mM to about 15 mM, about 0.5 mM to about 20 mM, about 1 mM to about 25 mM, about 2 mM to about 25 mM, about 3 mM to about 25 mM, about 5 mM to about 25 mM, about 10 mM to about 25 mM, about 15 mM to about 25 mM, about 20 mM to about 25 mM, about 1 mM to about 15 mM, or about 5 mM to about 10 mM). In some embodiments, a salt can be present in a polymer composition at a concentration of about 1 mM to about 15 mM (e.g., about 5 mM to about 10 mM (e.g., about 5.8 mM)).

In some embodiments, a polymer composition can include any appropriate additional components. In some embodiments, a polymer composition can further include one or more of an antimicrobial, a detergent, a stabilizer (e.g., an antioxidant), a chelator, a tonicity agent, a mucoadhesive, a preservative, a carrier, a penetration enhancer, or a combination thereof.

The term “stabilizer” can refer to compounds such as antioxidants that improve the compatibility of excipients with a container, or a delivery system, such as a syringe or a glass bottle, improve the stability of a component of a polymer composition, or improve overall stability of a polymer composition.

Tonicity and pH adjusting agents may also be included in a polymer composition, in some cases. In general, the endolymph has a higher osmolality than the perilymph. For example, the endolymph typically has an osmolality of about 304 mOsm/kg H₂O, while the perilymph typically has an osmolality of about 294 mOsm/kg H₂O. In some cases, a polymer composition is isotonic with the perilymph. Isotonic polymer compositions are typically provided by the addition of a tonicity agent. Suitable tonicity agents include, but are not limited to, a pharmaceutically acceptable sugar, salt (e.g., those disclosed herein) or any combinations or mixtures thereof, such as, but not limited to, dextrose, glycerin, mannitol, sorbitol, sodium chloride, and other electrolytes.

In some embodiments, a polymer composition can include a mucoadhesive. In some cases, the mucoadhesive facilitates adhesion to a portion of the ear, such as the external mucous layer of the round window membrane. Mucoadhesive agents include, but are not limited to, carbomers, such as CARBOPOL® 934P, polyvinylpyrrolidone polymer (PVP); a water-swellable, but water-insoluble, fibrous, cross-linked carboxy-functional polymer; a crosslinked poly(acrylic acid) (e.g. CARBOPOL® 947P); a carbomer homopolymer; a carbomer copolymer; a hydrophilic polysaccharide gum; maltodextrin; a cross-linked alginate gum gel, hydroxypropyl methylcellulose, and a water-dispersible polycarboxylated vinyl polymer. Some exemplary mucoadhesive agents are described in U.S. Pat. No. 8,828,980 to Lichter, et al.

A polymer composition may include penetration enhancers that allow, for example, delivery of the agents across a barrier, such as the oval window or the round window of the ear. Typically, the penetration enhancers are auris-compatible. Exemplary penetration enhancers include sodium lauryl sulfate, sodium octyl sulfate, sodium dodecyl sulfate, ocytl-trimethyl-ammonium bromine, dodecyl-trimethyl ammonium bromide, sodium laurate, polyoxyethylene-20-cetyl ether, laureth-9, sodium dodecylsulfate, dioctyl sodium sulfosuccinate, polyoxyethylene-9-lauryl ether (PLE), TWEEN® 20, TWEEN® 80, nonylphenoxypolyethylene (NP-POE), polysorbates, bile salts, fatty acids and derivatives, chelating agents(such as EDTA, citric acid, and salicylates, sulfoxides (such as dimethyl sulfoxide (DMSO) and decylmethyl sulfoxide), and alcohols (such as ethanol, isopropanol, glycerol, and propanediol.

In some forms, a polymer composition can include a preservative. Suitable preservatives include, but are not limited to, benzoic acid, boric acid, p-hydroxybenzoates, alcohols, quaternary compounds, stabilized chlorine dioxide, mercurials, such as merfen and thiomersal, or a combination thereof. Exemplary preservatives are described in U.S. Pat. No. 8,828,980 to Lichter, et al.

Typically, the polymer compositions also include water, and in some embodiments, the balance of the polymer composition is water.

A polymer composition can be prepared by any appropriate method. In some embodiments, a polymer composition can be prepared by a method including dissolving an active agent in PEG 300 to form an active agent solution, and adding to the active agent solution an aqueous solution comprising the poloxamer 407, and the poloxamer 188.

In some embodiments, a polymer composition can be prepared and stored in vials, syringes, capsules, ampules, or pouches prior to administration. In some embodiments, a polymer composition may be packaged in a single-dose that is administered intra-tympanically into the middle ear.

Polymer compositions as provided herein can have any appropriate properties.

A polymer composition can have a viscosity suitable for delivery (e.g., of an active agent) to the middle and/or inner ear. In some embodiments, a polymer composition can have a viscosity suitable for injection through a 23-G needle, for example, for injection through the tympanic membrane into the tympanic cavity. In some embodiments, 200 μL can be passed through a 25 G×1½ inch needle in less than 1 minute (e.g., less than 50 seconds, less than 40 seconds, or less than 30 seconds). In some embodiments, 0.5 mL of the polymer composition can be aspirated into a 1 mL pipette tip in less than 10 seconds (e.g., less than 9 seconds, less than 8 seconds, less than 7 seconds, less than 6 seconds, or less than 5 seconds).

A polymer composition can have a gelation temperature suitable for forming a gel in the middle and/or inner ear. In some embodiments, a polymer composition can form a gel at a temperature of about 24° C. to about 32° C. (e.g., about 25° C. to about 31° C. (e.g., about 26° C. to about 30° C.)). Formation of a gel can be determined by any appropriate method, such as a vial inversion test.

A polymer composition (e.g., including an active agent) can have a stability suitable for use as a pharmaceutical composition for delivery (e.g., of the active agent) to the middle and/or inner ear. Without being bound by any particular theory, it is believed that pharmaceutical compositions, after they are made, are often subject to shipping and/or storage before administration to a subject. It is therefore beneficial if a polymer composition (e.g., including an active agent) is physically stable for a period of time. Stability of a polymer composition (e.g., including an active agent) can be determined using any appropriate method. One method is to monitor the appearance of a polymer composition including an active agent for turbidity, precipitation, or both, for example, using visual inspection (e.g., under 5× magnification). Typically, if turbidity or precipitation is observed, the polymer composition including an active agent is no longer considered to be physically stable. This method may be particularly useful if an active agent is poorly soluble, as, without being bound by any theory, it is believed that a poorly soluble active agent may be prone to precipitating out of solution. As another example, physical stability of a solution can be determined by filtering the solution (e.g., removing precipitates) and measuring the concentration of a dissolved solute. Another method of measuring physical stability of a solution is to use light scattering, which can detect the presence of particulate matter.

Physical stability can be determined at any appropriate temperature, but some typical conditions include near-ambient temperatures (e.g., about 22° C. to about 28° C., or about 25° C.) and cool temperatures (e.g., about 2° C. to about 8° C., or about 5° C.). In some embodiments, a polymer composition (e.g., including an active agent) is physically stable for at least 3 days (e.g., at least 7 days, at least 14 days, at least 21 days, at least 28 days, at least 30 days, at least 56 days, at least 60 days, at least 1 month, at least 2 months, at least 3 months, at least 4 months, at least 6 months, at least 8 months, at least 10 months, at least 1 year, at least 2 years, or at least 3 years) at near ambient temperatures. In some embodiments, a polymer composition (e.g., including an active agent) is physically stable for at least 3 days (e.g., e.g., at least 7 days, at least 14 days, at least 21 days, at least 28 days, at least 30 days, at least 56 days, at least 60 days, at least 1 month, at least 2 months, at least 3 months, at least 4 months, at least 6 months, at least 8 months, at least 10 months, at least 1 year, or at least 2 years, or at least 3 years), and up to 5 years at near ambient temperatures. In some embodiments, a polymer composition (e.g., including an active agent) is physically stable for at least 3 days (e.g., e.g., at least 7 days, at least 14 days, at least 21 days, at least 28 days, at least 30 days, at least 56 days, at least 60 days, at least 1 month, at least 2 months, at least 3 months, at least 4 months, at least 6 months, at least 8 months, at least 10 months, at least 1 year, or at least 2 years, or at least 3 years), and up to 10 years at near ambient temperatures. In some embodiments, a polymer composition (e.g., including an active agent) is physically stable for at least 3 days (e.g., e.g., at least 7 days, at least 14 days, at least 21 days, at least 28 days, at least 30 days, at least 56 days, at least 60 days, at least 1 month, at least 2 months, at least 3 months, at least 4 months, at least 6 months, at least 8 months, at least 10 months, at least 1 year, or at least 2 years, or at least 3 years) at cool temperatures. In some embodiments, a polymer composition (e.g., including an active agent) is physically stable for at least 3 days (e.g., e.g., at least 7 days, at least 14 days, at least 21 days, at least 28 days, at least 30 days, at least 56 days, at least 60 days, at least 1 month, at least 2 months, at least 3 months, at least 4 months, at least 6 months, at least 8 months, at least 10 months, at least 1 year, or at least 2 years, or at least 3 years), and up to 5 years at cool temperatures. In some embodiments, a polymer composition (e.g., including an active agent) is physically stable for at least 3 days (e.g., e.g., at least 7 days, at least 14 days, at least 21 days, at least 28 days, at least 30 days, at least 56 days, at least 60 days, at least 1 month, at least 2 months, at least 3 months, at least 4 months, at least 6 months, at least 8 months, at least 10 months, at least 1 year, or at least 2 years, or at least 3 years), and up to 10 years at cool temperatures.

In some embodiments, a polymer composition disclosed herein can have a degree of supersaturation of about 1.0 to about 2.5. As used herein, a “degree of supersaturation” as the formulation strength (e.g., in mg/mL) divided by the dissolved concentration of the active agent, after being equilibrated with added crystals (e.g., 0.05 mg/mL) of the active agent. In some embodiments, a polymer composition disclosed herein can have a degree of supersaturation of about 1.0 to about 2.5 (e.g., about 1.0 to about 2.0, about 1.0 to about 1.5, about 2.0 to about 2.5, or about 1.5 to about 2.5) after 1 day at 15° C. In some embodiments, a polymer composition disclosed herein can have a degree of supersaturation of about 1.0 to about 2.5 (e.g., about 1.0 to about 2.0, about 1.0 to about 1.5, about 2.0 to about 2.5, or about 1.5 to about 2.5) after 1 day at 4° C.

Also provided herein are gels formed by any of the polymer compositions disclosed herein.

In some embodiments, a gel formed from a polymer composition provided herein can provide, for example, sustained release of an active agent for a period of at least 3-15 days in the ear. In some embodiments, a hydrogel formed from such a formulation can provide, for example, sustained release of an active agent for an extended period of time (e.g., one day, 2 days, 3 days, 4 days, 5 days, 6 days, 1 week, 2 weeks, 3 weeks, 1 month, 2 months, 3 months, 4 months, 5 months, or 6 months).

Also provided herein are methods of treating an otic disease or disorder in a subject including administering to the subject any of the polymer compositions disclosed herein. In some embodiments, provided herein is a method of treating an otic disease or disorder a subject in need thereof, the method including administering to an affected ear of the subject a therapeutically effective amount of a polymer composition (e.g., including an active agent) disclosed herein. In some embodiments, provided herein is a method of treating an otic disease or disorder in a subject, the method including: (a) identifying the subject as having the otic disease or disorder; and (b) administering to an affected ear of the subject a therapeutically effective amount of a polymer composition (e.g., including an active agent) disclosed herein.

An otic disease or condition can be any appropriate otic disease or condition, such as Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof.

Identifying a subject with an otic disease or condition can be performed using any appropriate method. In some embodiments, identifying a subject with an otic disease or condition includes identifying one or more symptoms of an otic disease or condition in the subject, such as vertigo (e.g., having two episodes of vertigo, each lasting 20 minutes or longer but not longer than 12 hours), hearing loss (e.g., verified by a hearing test), tinnitus or a feeling of fullness in the ear, a combination thereof, or all thereof. In some embodiments, an otic disease or condition is identified when a subject exhibits one or more symptoms of an otic disease or condition and other known causes of the symptoms are excluded. In some embodiments, identifying a subject with an otic disease or condition includes identifying presence of BLB leakage (e.g., using contrast enhanced MRI). In some embodiments, after a subject is identified as having an otic disease or condition, the presence of BLB leakage is identified (e.g., using contrast enhanced MRI). For example, MM can be used to detect hydrops and/or perilymphatic enhancement (which can often be considered to be a confirmatory sign of BLB leakage).

Polymer compositions are typically administered to the middle and/or inner ear of a subject in need thereof. In general, methods of use involve administering to the subject (e.g., by injection, such as intratympanic injection) compositions containing an effective amount of an active agent.

In some cases, a polymer composition (e.g., including an active agent) can also be used to treat other diseases which require a longer duration for treatment, such as through administration via a reservoir or depot.

In some embodiments, a polymer composition can be administered via localized administrations by intra-tympanic injection of the polymer composition (e.g., at room temperature or lower). After administration, a polymer composition may effect a transition from a liquid state at room temperature to a gel state at body temperature. In some embodiments, the gel state provides sustained release of an active agent.

In some embodiments, a polymer composition may also be administered on or near the round window or the crista fenestrae cochleae through entry via a post-auricular incision and surgical manipulation into or near the round window or the crista fenestrae cochleae area.

In some embodiments, administration is made using a syringe and small gauge needle, 23 G to 30 G or higher gauge, such as when a needle is inserted through the tympanic membrane. In some such embodiments, the polymer composition can fill the hypotympanum of the tympanic cavity, and contact the round window membrane.

In some cases, a polymer composition can also be administered into the tympanic cavity or applied on the tympanic membrane or onto or in the cochlea by injection, direct instillation or perfusion of the inner ear compartments, for example, via surgical procedures. In some instances, a polymer composition may be directly injected into the cochlea, for example, via injection through the round window membrane or a cochleostomy drilled in the bone of the cochlea.

In other embodiments, a polymer composition is administered via microcathethers implanted into the subject, using a drug delivery device such as a micropump, a microinjection device, or a microreservoir implanted within the inner ear.

Polymer compositions can be administered in a single dose or in multiple doses. Certain factors may influence the dosage required to effectively treat or prevent a disorder, including, but not limited to, the severity of the disease or disorder, previous preventions, the general health and/or age of the subject, and other diseases present. It will also be appreciated that the effective dosage of the composition used for prevention may increase or decrease over the course of a particular treatment time period. Need for changes in dosage quantity or strength may result become apparent from the results of assays, for example, frequency of vertigo attacks, tinnitus, the auditory brainstem response, distortion product otoacoustic emission, word recognition scores, and/or subjective changes in balance or hearing reported by the patient.

Polymer compositions can be administered to the middle ear of a subject in need thereof, for example, by transtympanic injection. In some embodiments, a polymer composition is administered on or near the round window membrane via transtympanic injection. Polymer compositions, in some embodiments, may also be administered on or near the round window or the crista fenestrae cochleae through entry via a post-auricular incision and surgical manipulation into or near the round window or the crista fenestrae cochleae area.

In some cases, administering can include using a syringe and small gauge needle, (e.g., 23 G to 30 G or smaller), wherein the needle is inserted through the tympanic membrane and guided to the area of the round window or crista fenestrae cochleae. The polymer composition is then deposited on or near the round window or crista fenestrae cochleae.

In some embodiments, a polymer composition can also be administered into the tympanic cavity or applied on the tympanic membrane or onto or in the cochlea by injection, direct instillation or perfusion of the inner ear compartments, or in surgical procedures including, cochleostomy, labyrinthotomy, mastoidectomy, stapedectomy, or endolymphatic sacculotomy.

The administering can include administering a therapeutically effective dose. In some embodiments, administering can include administering between about 5 and about 500 microliters (e.g., between about 5 μL and about 400 μL, between about 5 μL and about 300 μL, between about 5 μL and about 200 μL, between about 5 μL and about 100 μL, between about 5 μL and about 50 μL, between about 5 μL and about 25 μL, between about 5 μL and about 10 μL, between about 10 μL and about 500 μL, between about 25 μL and about 200 μL, between about 25 μL and about 500 μL, between about 50 μL and about 500 μL, between about 100 μL and about 500 μL, between about 200 μL and about 500 μL, between about 300 μL and about 500 μL, between about 400 and about 500 μL, between about 25 μL and about 300 μL, or between about 50 μL and about 200 μL) of a polymer composition, such as any of the polymer compositions provided herein. In some embodiments, administering can include administering about 50 μL, about 100 μL, or about 200 of a polymer composition, such as any of the polymer composition provided herein.

### EXAMPLES

### Example 1

### Preparation of Polymer Compositions

The polymer compositions as shown in Table 1 were prepared. The active agent was LPT99 (2-(4-(2,4-Dichlorophenethyl)-3,6-dioxo-1-(2-(thiophen-2-yl)ethyl)piperazin-2-yl)-N-(2-(5-methoxy-1H-indol-3-yl)ethyl)acetamide), which is poorly soluble, with a solubility in water of about 2.4 μM. The formulations were prepared by dissolving the active agent in the PEG300, then adding an aliquot of the PEG300 solution to a cool solution of the other components. In addition to the components shown, the compositions typically include a phosphate buffer (about 1 mM to about 5 mM (e.g., 2 mM)), and sodium chloride (about 5 mM to about 10 mM (e.g., 7 mM)).

TABLE 1
Polymer Compositions

| | Active Agent | P407 (% | P188 (% | PVP (% | PEG 300 (% |
|---|---|---|---|---|---|
| Comp. | (% by weight) | by weight) | by weight) | by weight) | by weight) |
| C1 | 0.05% | 15.00% | 0.00% | 0.00% | 0.00% |
| C2 | 0.05% | 15.00% | 10.00% | 0.00% | 10.00% |
| C3 | 0.05% | 15.00% | 10.00% | 2.50% | 10.00% |
| C4 | 0.05% | 16.40% | 0.00% | 0.00% | 9.10% |
| C5 | 0.05% | 16.40% | 0.00% | 2.50% | 9.10% |
| C6 | 0.05% | 16.67% | 0.00% | 0.00% | 5.00% |
| C7 | 0.05% | 16.67% | 0.00% | 1.00% | 5.00% |
| C8 | 0.05% | 16.67% | 0.00% | 2.50% | 5.00% |
| C9 | 0.05% | 16.67% | 0.00% | 0.00% | 5.00% |
| C10 | 0.05% | 16.67% | 0.00% | 0.00% | 9.00% |
| C11 | 0.05% | 16.67% | 0.00% | 1.00% | 9.00% |
| C12 | 0.05% | 16.67% | 0.00% | 2.50% | 9.00% |
| C13 | 0.05% | 16.67% | 2.50% | 0.00% | 9.00% |
| C14 | 0.05% | 16.67% | 2.50% | 0.00% | 13.00% |
| C15 | 0.05% | 16.67% | 5.00% | 0.00% | 9.00% |
| C16 | 0.05% | 16.67% | 5.00% | 0.00% | 13.00% |
| C17 | 0.05% | 16.67% | 5.00% | 0.00% | 13.00% |
| C18 | 0.05% | 16.67% | 10.00% | 0.00% | 9.00% |
| C19 | 0.05% | 16.67% | 10.00% | 0.00% | 13.00% |
| C20 | 0.05% | 17.60% | 2.50% | 0.00% | 9.00% |
| C21 | 0.05% | 17.60% | 2.50% | 0.00% | 13.00% |
| C22 | 0.05% | 17.60% | 5.00% | 0.00% | 5.00% |
| C23 | 0.05% | 17.60% | 5.00% | 1.00% | 5.00% |
| C24 | 0.05% | 17.60% | 5.00% | 2.50% | 5.00% |
| C25 | 0.05% | 17.60% | 5.00% | 0.00% | 9.00% |
| C26 | 0.05% | 17.60% | 5.00% | 0.00% | 9.00% |
| C27 | 0.05% | 17.60% | 5.00% | 1.00% | 9.00% |
| C28 | 0.05% | 17.60% | 5.00% | 2.50% | 9.00% |
| C29 | 0.05% | 17.60% | 5.00% | 0.00% | 9.00% |
| C30 | 0.05% | 17.60% | 5.00% | 0.00% | 13.00% |
| C31 | 0.05% | 17.60% | 10.00% | 0.00% | 9.00% |
| C32 | 0.05% | 17.60% | 10.00% | 0.00% | 13.00% |
| C33 | 0.05% | 18.54% | 10.00% | 0.00% | 5.00% |
| C34 | 0.05% | 18.54% | 10.00% | 1.00% | 5.00% |
| C35 | 0.05% | 18.54% | 10.00% | 2.50% | 5.00% |
| C36 | 0.05% | 18.54% | 10.00% | 0.00% | 9.00% |
| C37 | 0.05% | 18.54% | 10.00% | 1.00% | 9.00% |
| C38 | 0.05% | 18.54% | 10.00% | 2.50% | 9.00% |

### Example 2

### Stability Evaluation of Polymer Compositions I

Compositions C1-C5 were evaluated for the ability to stabilize the active agent. The compositions were prepared as in Example 1 and stored between 2° C. and 8° C. for 14 days.

Visual inspection for precipitation was performed with using 5× magnification. Refrigerated samples were stored briefly during inspections in an ice-chilled metal block to avoid heating. Table 2 shows the results of this experiment. Accordingly, this data shows that addition of PEG300 alone did not prevent precipitation of the active agent.

TABLE 2
Stability evaluation of compositions C1-C5

| Composition | Observation |
|---|---|
| C1 | Active agent never dissolved |
| C2 | Clear at 14 days |
| C3 | Clear at 14 days |
| C4 | Settled precipitant observed at 6 days |
| C5 | Hint of settled precipitant observed at 9 days |

### Example 3

### Accelerated Physical Stability Evaluation of Polymer Compositions

It can take long and variable amounts of time before drug may precipitate in a metastable formulation, typically due to the variable amount of time it takes to form drug crystal nuclei of critical size. In this test, that first part of the process was bypassed by adding drug crystals (0.05 mg/mL) to the formulation to serve as nucleation sites to accelerate the drug precipitation in metastable formulations. At predetermined times, the formulation was filtered (0.45 μm) and the dissolved drug concentration was measured in the supernatant. If the drug concentration did not decrease, the formulation was not supersaturated and precipitation should not occur during storage.

The results for 0% or 10% PVP were pooled since results showed no impact on crystal growth. Formulation C39 includes 14.7% Poloxamer 407, 0% Poloxamer 188, 0% PVP, 0% PEG 300, 0.5 mg/mL of active agent, 1.7 mM phosphate buffer, and 5.8 mM sodium chloride where the active agent was dissolved using ultrasound. Table 3 shows the results of this experiment.

TABLE 3
Nucleation evaluation of selected compositions.

| | | | | | C39 | C6 and C7 | C22 and C23 | C33 and C34 | C37 and C38 |
|---|---|---|---|---|---|---|---|---|---|
| | | | | | Days to precip. at 4° C. | | | | |
| | | | | | NT | 1 | 14 | >200 | >200 |
| | | | | | Days to precip. at 25° C. | | | | |
| | | | | | NT | >200 | >200 | >200 | >200 |
| Sample Description | Temp | | Time | | Active Agent (μg/mL) in supernatant | | | | |
| Active agent added | 10-12° | C. | 1 | hr | 130 | 454 | 474 | 481 | 500 |
| Active agent added | 10-12° | C. | 24 | hr | NT | 110 | 333 | 379 | 451 |
| No active agent added | RT | | 7 | wks | 499 | 453 | 444 | 449 | NT |
| No DS added | 4° | C. | 7 | wks | NT | 3.7 | 6 | NT | NT |

NT = Not tested

### Example 4

### Stability Evaluation of Polymer Compositions II

Selected compositions were evaluated for the ability to stabilize the active agent. The compositions were prepared as in Example 1 and stored either at “warm” temperatures (room temperature and/or 25° C.), or at “cool” temperatures (5° C. and/or 2-8° C.) for up to 2 years.

Visual inspection for precipitation was performed with using 5× magnification. Refrigerated samples were stored briefly during inspections in an ice-chilled metal block to avoid heating. Table 4 shows the results of this experiment. The state of the polymer composition is noted and each polymer composition had two or three replicates. Compositions marked with an asterisk (e.g., C8*) are control samples lacking the active agent. The following abbreviations are used:
C=Clear
T=Turbid
P=Precipitate
NI=Not inspected
D=appearance of droplets on bottom
S=solids on bottom, few, do not look fluid
SS=single solid speck

Since all control samples remained clear, observations of precipitate, droplets or solids are attributed to phase separation of active agent.

TABLE 4
Stability evaluation of selected polymer compositions

| | | | | | | | | | | | | |
|---|---|---|---|---|---|---|---|---|---|---|---|---|
| C6 | C7 | C8 | C8* | C10 | C11 | C12 | C12* | C22 | C23 | C24 | C24* | C25 |

Warm Samples

| | | | | | | | | | | | | | |
|---|---|---|---|---|---|---|---|---|---|---|---|---|---|
| RT Day 1 | C | C | C | C | C | C | C | C | C | C | C | C | C |
| 25° C. | C | C | C | C | C | C | C | C | C | C | C | C | C |
| Day 3 | | | | | | | | | | | | | |
| RT Day 7 | C | C | C | C | C | C | C | C | C | C | C | C | C |
| 25° C. | C | C | C | C | C | C | C | C | C | C | C | C | C |
| Day 14 | | | | | | | | | | | | | |
| 25° C. | C | C | C | C | C | C | C | C | C | C | C | C | C |
| Day 22 | | | | | | | | | | | | | |
| 25° C. | C | C | C | C | C | C | C | C | C | C | C | C | C |
| Day 29 | | | | | | | | | | | | | |
| 25° C. | C | C | C | C | C | C | C | C | C | C | C | C | C |
| Day 37 | | | | | | | | | | | | | |
| 25° C. | C | C | C | C | C | C | C | C | C | C | C | C | C |
| Day 56 | | | | | | | | | | | | | |
| 25° C. | C | C | C | C | C | C | C | C | C | C | C | C | C |
| 4 Mos | | | | | | | | | | | | | |
| 25° C. | C, P | C, P | C, P | C | C, P | C, P | C, P | C | C, P | C, P | C | C | C |
| 2 yrs | | | | | | | | | | | | | |

Cool Samples

| | | | | | | | | | | | | | |
|---|---|---|---|---|---|---|---|---|---|---|---|---|---|
| 5° C. | T | T, P | T | C | T | C | C | C | C | C | C | C | C |
| Day 1 | | | | | | | | | | | | | |
| 5° C. | T, P | T, P | T, P | C | C, P | C | C | C | C | C | C | C | C |
| Day 3 | | | | | | | | | | | | | |
| 5° C. | T, P | T, P | T, P | C | C, P | C | C | C | C | C | C | C | C |
| Day 7 | | | | | | | | | | | | | |
| 2-8° C. | C, P | C, P | C, P | C | C, P | C | C, P | C | C, P | C, P | C, P | C | C |
| Day 14 | | | | | | | | | | | | | |
| 2-8° C. | C, P | C, P | C, P | C | C, P | C | C, P | C | C, P | C, P | C, P | C | C |
| Day 22 | | | | | | | | | | | | | |
| 2-8° C. | C, P | C, P | C, P | C | C, P | C | C, P | C | C, P | C, P | C, P | C | C |
| Day 28 | | | | | | | | | | | | | |
| 2-8° C. | NI | NI | NI | NI | NI | T | C, P | C | NI | NI | NI | NI | C |
| Day 35 | | | | | | | | | | | | | |
| 2-8° C. | NI | NI | NI | NI | NI | P | NI | NI | NI | NI | NI | NI | C |
| Day 56 | | | | | | | | | | | | | |
| 2-8° C. | NI | NI | NI | NI | NI | P | NI | NI | NI | NI | NI | NI | D |
| Day 99 | | | | | | | | | | | | | |
| 2-8° C. | NI | NI | NI | NI | NI | NI | NI | NI | NI | NI | NI | NI | D, P |
| Day 100 | | | | | | | | | | | | | |
| 2-8° C. | NI | NI | NI | NI | NI | P | NI | NI | NI | NI | P | C | S, P |
| 4 mos | | | | | | | | | | | | | |
| 2-8° C. | NI | NI | NI | NI | NI | NI | NI | NI | NI | NI | NI | NI | C, P |
| 2 yrs | | | | | | | | | | | | | |

| | C27 | C28 | C28* | C33 | C33* | C34 | C35 | C35* | C36 | C37 | C38 | C38* |
|---|---|---|---|---|---|---|---|---|---|---|---|---|
| | Warm Samples | | | | | | | | | | | |
| RT Day 1 | C | C | C | C | C | C | C | C | C | C | C | C |
| 25° C. | C | C | C | C | C | C | C | C | C | C | C | C |
| Day 3 | | | | | | | | | | | | |
| RT Day 7 | C | C | C | C | C | C | C | C | C | C | C | C |
| 25° C. | C | C | C | C | C | C | C | C | C | C | C | C |
| Day 14 | | | | | | | | | | | | |
| 25° C. | C | C | C | C | C | C | C | C | C | C | C | C |
| Day 22 | | | | | | | | | | | | |
| 25° C. | C | C | C | C | C | C | C | C | C | C | C | C |
| Day 29 | | | | | | | | | | | | |
| 25° C. | C | C | C | C | C | C | C | C | C | C | C | C |
| Day 37 | | | | | | | | | | | | |
| 25° C. | C | C | C | C | C | C | C | C | C | C | C | C |
| Day 56 | | | | | | | | | | | | |
| 25° C. | C | C | C | C | C | C | C | C | C | C | C | C |
| 4 Mos | | | | | | | | | | | | |
| 25° C. | C | C | C | C | C | C | C | C | C | C | C | C |
| 2 yrs | | | | | | | | | | | | |
| | Cool Samples | | | | | | | | | | | |
| 5° C. | C | C | C | C | C | C | C | C | C | C | C | C |
| Day 1 | | | | | | | | | | | | |
| 5° C. | C | C | C | C | C | C | C | C | C | C | C | C |
| Day 3 | | | | | | | | | | | | |
| 5° C. | C | C | C | C | C | C | C | C | C | C | C | C |
| Day 7 | | | | | | | | | | | | |
| 2-8° C. | C | C | C | C | C | C | C | C | C | C | C | C |
| Day 14 | | | | | | | | | | | | |
| 2-8° C. | C | C | C | C | C | C | C | C | C | C | C | C |
| Day 22 | | | | | | | | | | | | |
| 2-8° C. | C | C | C | C | C | C | C | C | C | C | C | C |
| Day 28 | | | | | | | | | | | | |
| 2-8° C. | C | C | C | C | C | C | C | C | C | C | C | C |
| Day 35 | | | | | | | | | | | | |
| 2-8° C. | C | C | C | C | C | C | C | C | C | C | C | C |
| Day 56 | | | | | | | | | | | | |
| 2-8° C. | D | C | C | C | C | C | C | C | C | C | C | C |
| Day 99 | | | | | | | | | | | | |
| 2-8° C. | D | C | NI | NI | NI | NI | NI | NI | C | C | NI | NI |
| Day 100 | | | | | | | | | | | | |
| 2-8° C. | S | C, SS | C | C | C | C | C | C | C | C | C | C |
| 4 mos | | | | | | | | | | | | |
| 2-8° C. | C, P | C, P | C | C, P | C | C | C, P | C | C | C | C | C |
| 2 yrs | | | | | | | | | | | | |

### Example 5

### Functional and Stability Evaluation of Polymer Compositions

Selected polymer compositions were evaluated for functional and stability characteristics. Real-time stability evaluations were performed for limited duration, with visual inspection for precipitation using 5× magnification. Refrigerated samples were stored briefly during inspections in an ice-chilled metal block to avoid heating.

The primary physical stability assessments were accelerated physical stability evaluations performed as described in Example 3. The active ingredient concentration in the supernatant was determined by HPLC after 1 day equilibration and filtration. The Degree of Supersaturation was calculated, defined as the formulation strength (all had a formulation strength of approximately 550 mg/mL) divided by the dissolved concentration equilibrated with the added API crystals. The turbidity of the samples was assessed semi-quantitatively (least turbid=1, most turbid=4) since filtering may not remove all crystals if small in size in the samples with high turbidity.

“Gel to liquid temp” was determined by performing a vial inversion test. A vial containing approximately 0.5 mL of polymer composition was equilibrated at elevated temperature (e.g., 37° C.) to form a gel. Then the vials were inverted quickly and temperature was lowered stepwise (e.g., steps of 0.5° C. and then at least 5 minutes wait time) until visual observation of flow through the transparent window of an incubator.

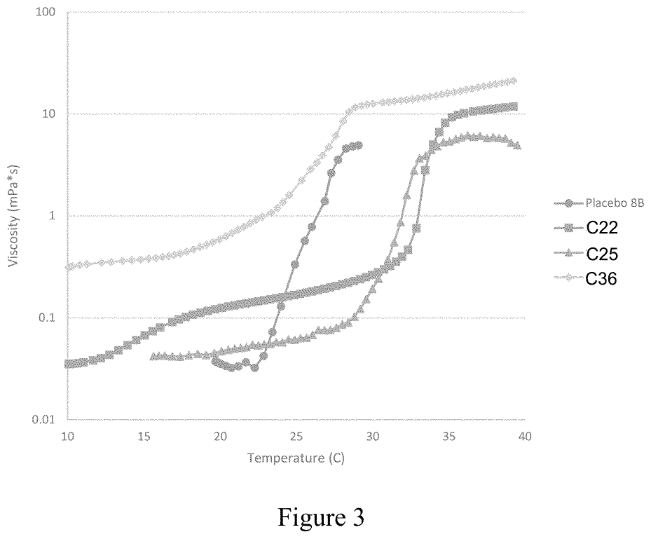

Viscosity was assessed by measuring the time to aspirate a polymer composition into a pipette tip. Select samples (placebo 8B, C22, C25, and C36) were also characterized in rheology testing (FIG. 3). The placebo 8B is a sample that contained no P188 or PEG300.

The results are shown in Table 5. All formulations in table below have 1.7 mM phosphate buffer and 5.8 mM sodium chloride.

TABLE 5
Functional and physical stability evaluation of polymer compositions

| | | | | | | | Gel to | Viscosity |
|---|---|---|---|---|---|---|---|---|
| | Accel Phys. | Accel Phys. | | | | | Liquid | comparison |
| | Stab. 1 day @ | Stab. 1 day @ | Degree of | Degree of | Accel Phys. | Phys. Stab. | Temp. | (Seconds to |
| | 15° C. | 4° C. | Supersat. 1 | Supersat. 1 | Stab. 1 day @ | 14 days @ | (vial inv) | aspirate 0.5 |
| | (mg/mL) | (mg/mL) | day @ 15° C. | day @ 4° C. | 4° C. Turbidity | 4° C. | (° C.) | mL into tip) |
| C9 | 170 | 385 | 3.3 | 1.5 | 4 | N | <28 | 4 |
| C13 | 406 | 85 | 1.3 | 6.3 | 4 | N | 33.0 | 5 |
| C14 | 437 | 257 | 1.2 | 2.0 | 3 | Y | 31.5 | 5 |
| C15 | 464 | 207 | 1.2 | 2.7 | 3 | Y | 35.0 | 5 |
| C16 | 450 | 423 | 1.2 | 1.3 | 2 | Y | 33, 32 | 5 |
| C18 | 492 | 462 | 1.1 | 1.2 | 2 | Y | 32.0 | 7 |
| C19 | 515 | 508 | 1.1 | 1.1 | 2 | Y | 29.0 | 9 |
| C20 | 451 | 190 | 1.2 | 2.9 | 4 | N | 28.5 | 7 |
| C21 | 463 | 407 | 1.2 | 1.4 | 3 | Y | <28 | 7 |
| C26 | 459 | 288 | 1.3 | 2.0 | 2 | Y | 32.0 | 5 |
| C29 | 475 | 313 | 1.2 | 1.8 | 3 | Y | 31.5 | 7 |
| C30 | 495 | 518 | 1.1 | 1.1 | 2 | Y | 29.5 | 7 |
| C31 | 468 | 521 | 1.2 | 1.1 | 1 | Y | 29.0 | 7 |
| C32 | 540 | 540 | 1.0 | 1.0 | 1 | Y | <28 | 10 |

### Example 6

### Injectability Evaluation of Polymer Compositions

Several formulations were chosen and provided to an ENT surgeon for evaluation of injectability through various gauge needles typically used for intratympanic injection. The tests were carried out at ambient temperature of 22° C. All formulations tested were considered to be acceptable for intratympanic injection. Table 6 shows the time for injection of 200 μL from the ENT surgeon.

TABLE 6
Time for injection

| | 25G × 1 1/2 in | Spinal (25G × 3 1/2 in) |
|---|---|---|
| C15 | 20 seconds | |
| C18 | 22 seconds | |
| C25 | 22 seconds | |
| C29 | 26 seconds | |
| C32 | 48 seconds | |
| C36 | 28.8 seconds | 44 seconds |

### Example 7

### Drug Release Evaluation of Gelled Polymer Compositions

The rate of drug release from a gel formed by replicates of polymer composition C36 was determined to be, on average, 24.4 percent cumulative release/hr^{1/2}.

Testing was done with vertical Franz Cells (PermeGear 6 G-01-00-09-02-VD-0725-FL) and a 0.2 um porous polycarbonate membrane (Whatman Cat. No. 110606) intended to keep the drug product sample and receptor medium separate with minimal resistance to the drug release rate. Drug was released from 0.4 mL of drug product into the 2.0 volume receptor chambers containing PBS with 5% P407 added to increase drug solubility. Testing was performed with Franz Cells on a stir plate inside an incubator at 37° C.

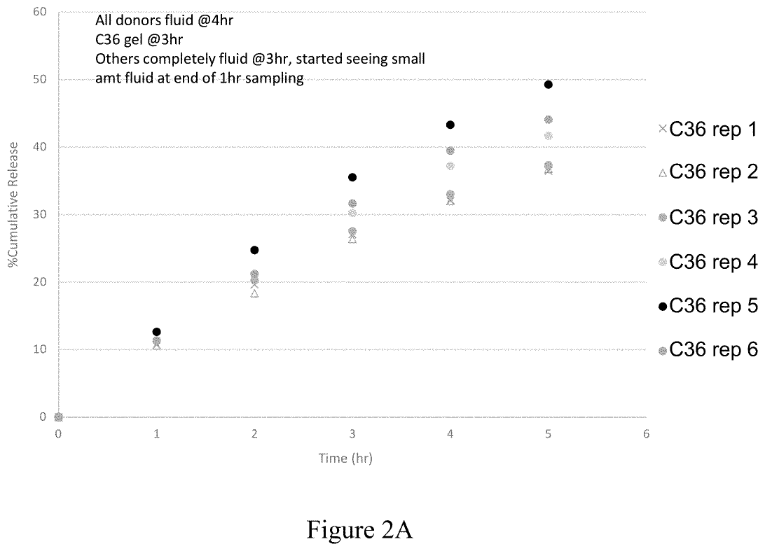

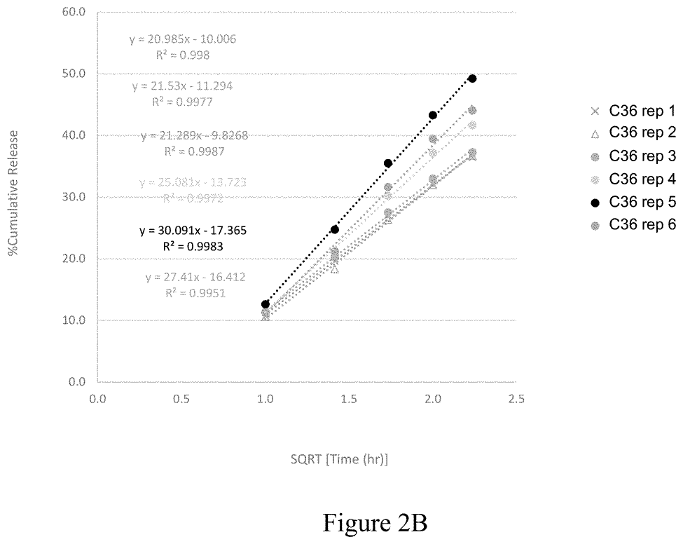

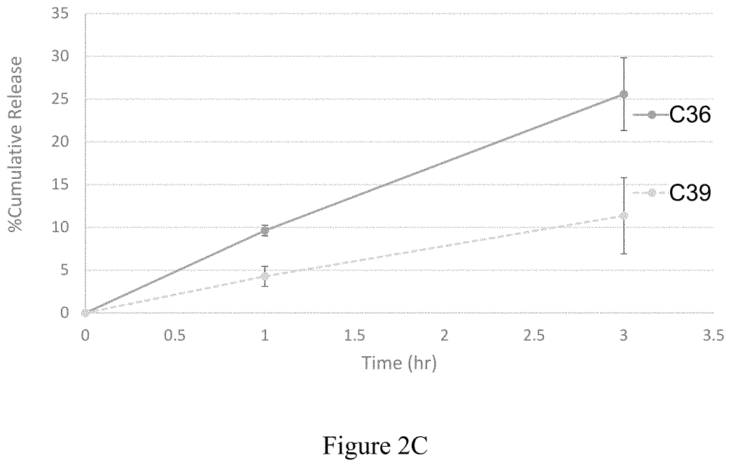

The percent cumulative release is plotted in FIG. 2A (against time, in hours) and FIG. 2B (against the square root of the time in hours). The repeatability of the test method was evaluated by assessing sameness of the drug release rates as described in USP 1724. FIG. 2C compares the percent cumulative release of an active agent from a gel formed by polymer composition C36 and control polymer composition C1 (manufactured with ultrasound to dissolve the API).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. (canceled)

2. A polymer composition comprising:
about 15% to about 22% by weight of poloxamer 407;
about 1% to about 12% by weight of poloxamer 188; and
about 3% to about 15% by weight of PEG 300;
provided that:
(a) if the amount of poloxomer 188 is less than 3% by weight, the amount of PEG 300 is about 13% to about 15% by weight,
(b) if the amount of poloxamer 188 is about 3% to about 5% by weight and the amount of poloxamer 407 is less than about 17% by weight, the amount of PEG 300 is about 13% to about 15% by weight,
(c) if the amount of poloxamer 188 is about 3% to about 5% by weight and the amount of poloxamer 407 is at least about 17% by weight, the amount of PEG 300 is about 9% to about 15% by weight, and
(d) if the amount of poloxamer 188 is about 10% to about 12% by weight, the amount of PEG 300 is about 3% to about 10% by weight.

3. The polymer composition of claim 2, wherein the total amount of poloxamer 188 and PEG300 in the composition is about 12% to about 25% by weight.

4-5. (canceled)

6. The polymer composition of claim 2, wherein the amount of poloxamer 407 is about 16% to about 19% by weight.

7-9. (canceled)

10. The polymer composition of claim 2, wherein the amount of poloxamer 188 is about 1% to about 12% by weight.

11. The polymer composition of claim 2, wherein the amount of poloxamer 188 is about 1% to about 3% by weight.

12. The polymer composition of claim 11, wherein the amount of PEG 300 is about 13% to about 15% by weight.

13. The polymer composition of claim 2, wherein the amount of poloxamer 188 is about 3% to about 5% by weight.

14. The polymer composition of claim 13, wherein the amount of poloxamer 407 is less than about 17% by weight and the amount of PEG 300 is about 13% to about 15% by weight.

15. The polymer composition of claim 13, wherein the amount of poloxamer 407 is at least about 17% by weight and the amount of PEG 300 is about 9% to about 15% by weight.

16-17. (canceled)

18. The polymer composition of claim 17, wherein the amount of PEG 300 is about 3% to about 13% by weight.

19-26. (canceled)

27. A polymer composition comprising:
about 17% to about 20% by weight of poloxamer 407;
about 9% to about 11% by weight of poloxamer 188;
about 8% to about 10% by weight of PEG 300; and
about 0.01% to about 1.0% by weight of an active agent.

28. (canceled)

29. The polymer composition of claim 27, wherein the active agent has a solubility in water of less than about 100μM.

30-34. (canceled)

35. The polymer composition of claim 27, wherein the active agent has a melting temperature of at least 125° C.

36-38. (canceled)

39. The polymer composition of claim 27, comprising about 0.03% to about 0.07% by weight of the active agent.

40. (canceled)

41. The polymer composition of claim 27, further comprising about 0.5% to about 3% of polyvinylpyrrolidone.

42. (canceled)

43. The polymer composition of claim 27, further comprising about 0.5 mM to about 10 mM of a buffer selected from the group consisting of a phosphate buffer, a tris(hydroxymethyl)aminomethane buffer, a citrate buffer, an acetate buffer, a 4-(2-hydroxyethyl)-1-piperazineethanesulfonic acid buffer, or a combination thereof.

44-48. (canceled)

49. The polymer composition of claim 27, further comprising about 1 mM to about 15 mM of a salt selected from the group consisting of sodium chloride, potassium chloride, calcium chloride, magnesium chloride, sodium thiosulfate, sodium bisulfite, sodium bicarbonate, ammonium sulfate, and combinations thereof.

50. (canceled)

51. The polymer composition of claim 27, wherein the active agent is physically stable in the composition for at least 14 days at 4° C.

52-62. (canceled)

63. The polymer composition of claim 27, wherein the polymer composition forms a gel at a temperature of about 24° C. to about 32° C.

64-67. (canceled)

68. A method of treating an otic disease or disorder in a subject in need thereof, the method comprising administering to an affected ear of the subject a therapeutically effective amount of a polymer composition comprising:
about 17% to about 20% by weight of poloxamer 407;
about 9% to about 11% by weight of poloxamer 188;
about 8% to about 10% by weight of PEG 300; and
about 0.01% to about 1.0% by weight of an active agent.

69-72. (canceled)
